(19) United States
(12) Patent Application Publication
(10) Pub. No.: US 2026/0240634 A1
(43) Pub. Date: Aug. 20, 2026

(54) MALE INCONTINENCE CLAMP SYSTEM AND METHOD

(71) Applicant: Cross Innovations LLC, Cheyenne, WY (US)

(72) Inventor: Shawn P Higgins, CHEYENNE, WY (US)

(21) Appl. No.: 19/641,989

(22) Filed: Apr. 8, 2026

Related U.S. Application Data
(63) Continuation of application No. 19/538,800, filed on Feb. 12, 2026.
Application No. 19/538,800 is a continuation of application No. 18/669,244, filed on May 20, 2024.

(51) Int. Cl. A61F 2/00

(52) U.S. Cl. CPC A61F 2/0054; A61F 2220/0091; A61F 2230/0013

(57) ABSTRACT

A method of using a male incontinence clamp to address incontinence in a user that includes applying the male incontinence clamp to a male member of the user. The male incontinence clamp comprising a first bar, a second bar, a rotatable adjustment assembly coupled to the second bar, a member cavity, and one or more sliders configured to adjust the fit of the male incontinence clamp including allowing the volume of the member cavity to be increased and decreased.

### CROSS-REFERENCE TO RELATED APPLICATIONS

This application is a continuation of U.S. Application No. 19/538,800, filed Feb. 12, 2026, entitled “MALE INCONTINENCE CLAMP SYSTEM AND METHOD,” with attorney docket number 0122777-004US1, which is a continuation of U.S. Application No. 18/669,244, filed May 20, 2024, entitled “MALE INCONTINENCE CLAMP SYSTEM AND METHOD,” with attorney docket number 0122777-004US0. These applications are hereby incorporated herein by reference in their entirety and for all purposes.

This application is related to U.S. Design Patent Application No. 29/943,159, filed May 20, 2024, entitled “Clamp,” with attorney docket number 0122777-005US0. This application is hereby incorporated herein by reference in its entirety and for all purposes.

### BRIEF DESCRIPTION OF THE DRAWINGS

FIG. 1a is an example first perspective view illustrating an embodiment of a male incontinence clamp.

FIG. 1b is an example second perspective view illustrating an embodiment of a male incontinence clamp.

FIG. 2a is an example top view illustrating an embodiment of a male incontinence clamp.

FIG. 2b is an example first side view illustrating an embodiment of a male incontinence clamp.

FIG. 2c is an example second side view illustrating an embodiment of a male incontinence clamp.

FIG. 3 is an exploded perspective view illustrating a portion of an embodiment of a male incontinence clamp.

FIG. 4a illustrates an embodiment of an adjustment cylinder in a first configuration.

FIG. 4b illustrates an embodiment of the adjustment cylinder of FIG. 4a in a second configuration.

FIGS. 5 is an exploded perspective view illustrating a portion of an embodiment of a male incontinence clamp.

FIG. 6 is a cross-sectional view of an embodiment of a male incontinence clamp.

FIG. 7a illustrates an example anatomical cross section of a male member.

FIG. 7b illustrates a side view of an embodiment of a male incontinence clamp.

FIG. 8 illustrates a block diagram of an example method of using a male incontinence clamp.

It should be noted that the figures are not drawn to scale and that elements of similar structures or functions are generally represented by like reference numerals for illustrative purposes throughout the figures. It also should be noted that the figures are only intended to facilitate the description of the preferred embodiments. The figures do not illustrate every aspect of the described embodiments and do not limit the scope of the present disclosure.

### DETAILED DESCRIPTION OF THE PREFERRED EMBODIMENTS

Many men, especially older individuals, face the challenge of urinary incontinence, which is the uncontrolled and involuntary passage of urine. Incontinence can arise from various causes, including medical procedures, partial or complete sphincter loss, diseases, neurological dysfunctions, malformations of the urethral valve, and physical deterioration associated with aging, leading to the natural urethral valve or sphincter’s inability to control the flow of urine from the bladder. Regardless of the cause, incontinence is a significant problem that can lead to distress, embarrassment, inconvenience, and restriction of activities. One approach to preventing involuntary urine flow in incontinent males can be to clamp the penis using a device. However, the use of conventional devices often becomes painful, socially restrictive, and difficult to maintain hygiene.

The aforementioned approaches to addressing urinary incontinence in men leave much to be desired, as many designs offer little in terms of comfort or convenience for the user. Furthermore, it can be desirable to allow for fine tuning the pressure applied to the urethra. The following disclosure illustrates various preferred embodiments of a male incontinence clamp 100, that in some examples address one or more of these issues.

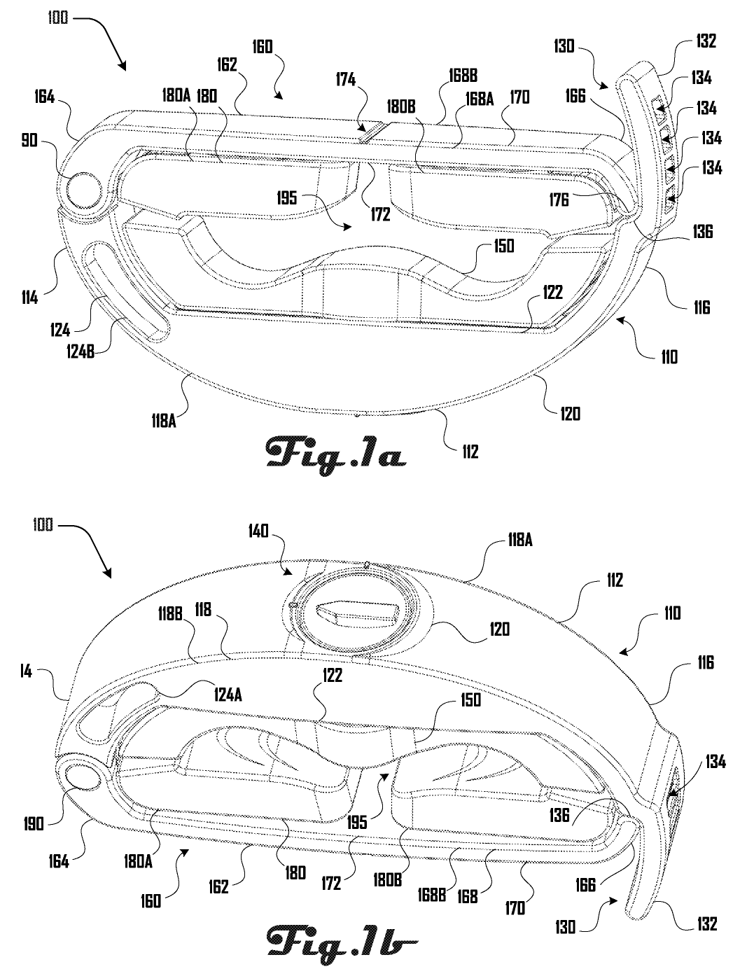

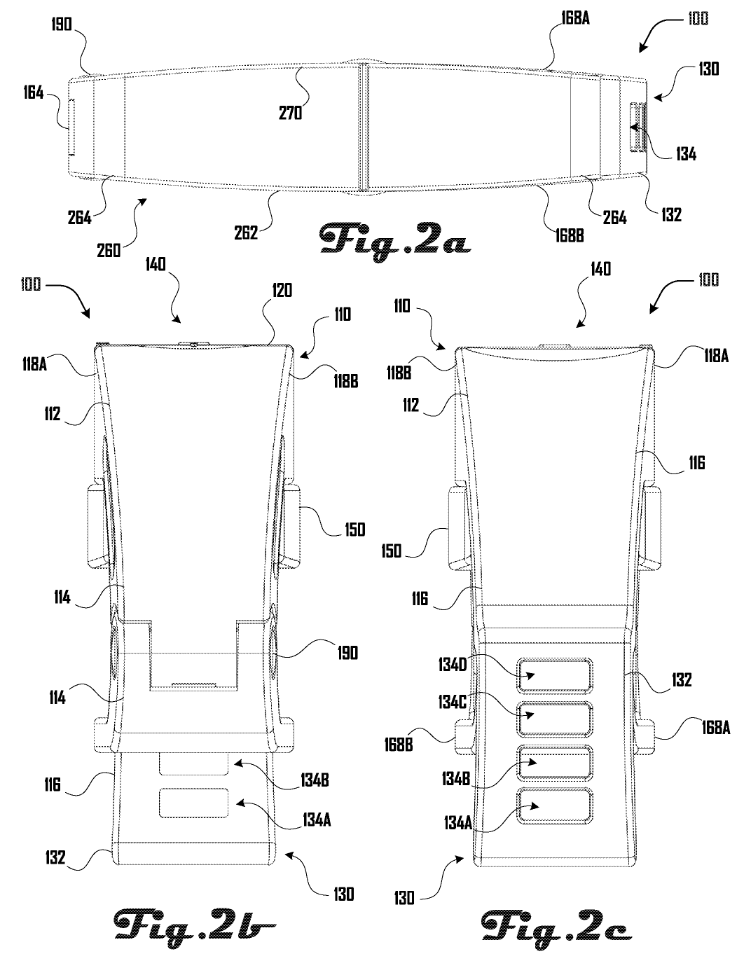

Turning to FIGS. 1a, 1b, 2a, 2b and 2c, an example embodiment of a male incontinence clamp 100 is illustrated that comprises a first bar 110 that is rotatably coupled to a second bar 160 via a hinge assembly 190 to define a member cavity 195. The first bar 110 includes a ratchet end 130, and an adjustment assembly 140 configured to adjust a first pad assembly 150. The second bar 160 comprises a second pad assembly 180 comprising a first and second pad 180A, 180B.

The first bar 110 in this example embodiment comprises a first bar central body 112 from which a first rear arm 114 and first front arm 116 extend. The first bar 110 defines a first and second first bar sidewall 118A, 118B, a first bar external face 120, a first bar internal face 122, and a pair of first bar slots 124A, 124B on opposing sides of the first bar 110. The first front arm 116 comprises a ratchet end 130 that defines a first arm tip 132, a plurality of ratchet slots 134, and stop notch 136. As discussed in more detail herein, the adjustment assembly 140 is configured to adjust the first pad assembly 150 disposed at the first bar internal face 122.

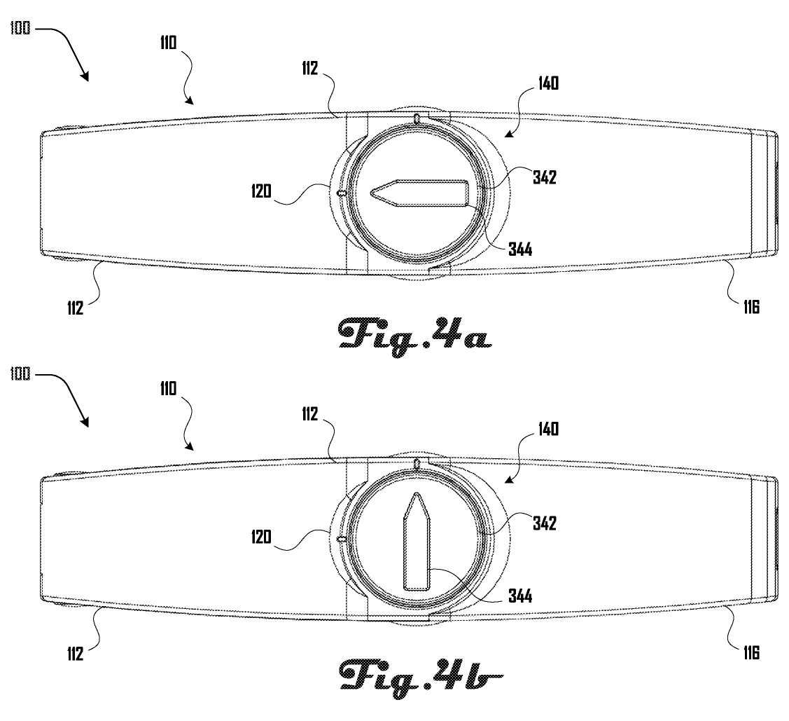

The first bar sidewalls 118 in various embodiments can be planar and disposed in parallel; angled relative to each other (e.g., as shown in FIGS. 2b and 2c); convexly arched in symmetry (e.g., as shown in FIGS. 4a and 4b), and the like. In various embodiments, the first bar 110 can have a concave arched configuration with the first bar external face 120 defined by the first bar central body 112, first rear arm 114 and first front arm 116 having an arched profile. The first bar external face 120 defined by the first bar central body 112 can be arched with the first bar internal face 122 defined by the first bar central body 112 being planar. The portions of the first bar external face 120 and first bar internal face 122 defined by the first rear arm 114 and first front arm 116 can be curved extending away from the first bar central body 112.

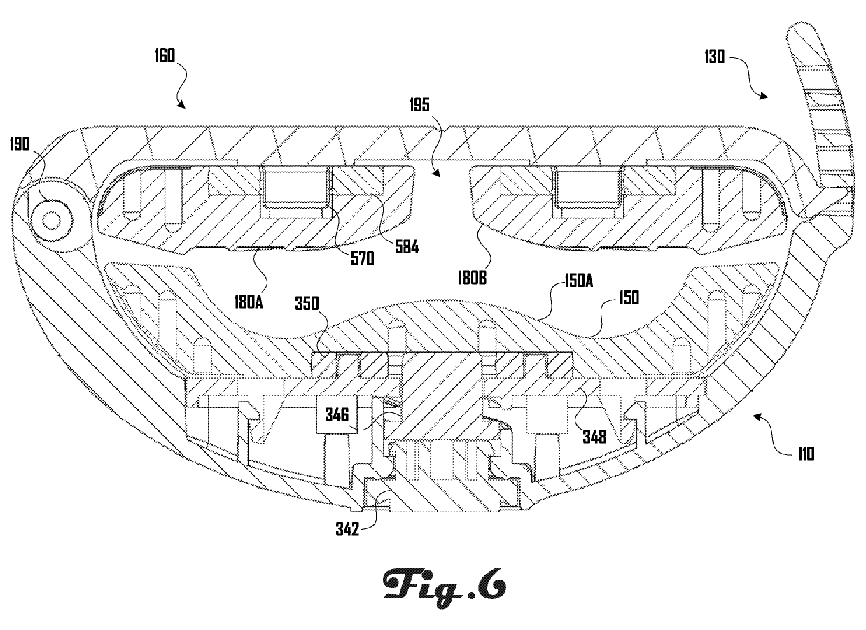

The second bar 160 in this example embodiment comprises a second bar central body 162 from which a second rear arm 164 and second front arm 166 extend. The second bar 160 defines a first second bar sidewall 168A and second second bar sidewall 168B, a second bar external face 170, and a second bar internal face 172 on which the first and second pads 180A, 180B are disposed. The second bar central body 162 of the second bar 160 defines a dorsal gap 174, which as discussed herein, can provide visual assistance with placement and alignment of the male incontinence clamp 100 on a male member. The second bar 160 can comprise a stop tip 176 at the end of the second front arm 166 which, as discussed herein, can be configured to engage with the stop notch 136 of the ratchet end 130 of the first bar 110 to generate a closure stop between the first bar 110 and the second bar 160. The stop tip 176 can further comprise a ratchet tooth 178 (see FIG. 6), which can be configured to engage with the ratchet slots 134 of the ratchet end 130 of the first bar 110, as discussed herein, to couple the first and second bars 110, 160 together.

The second bar sidewalls 168 in various embodiments can be planar and disposed in parallel; angled relative to each other; convexly arched in symmetry (e.g., as shown in FIG. 2a), and the like. In various embodiments, the second bar 160 can have a concave configuration with the second bar external and internal faces 170, 172 defined by second bar central body 162 being planar and disposed in parallel. The second arm rear end 164 and second arm front end 166 can be curved and extend away from the second bar central body 162.

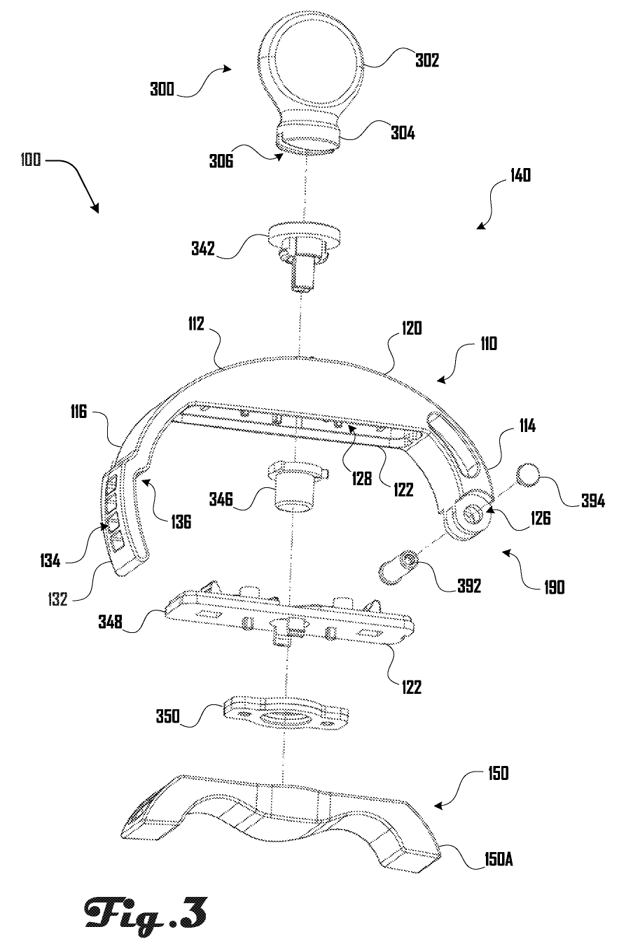

In some embodiments, the first bar 110 and second bar 160 can be rotatably coupled together via a hinge assembly 190, which in some examples can comprise a hinge 392 and hinge cap 394 (see e.g., FIG. 3) that extend through respective hinge ends 126, 179 of the first and second bars 110, 160 (see, e.g., FIGS. 3 and 6). However, it should be clear that the first bar 110 and second bar 160 can be rotatably coupled together in various suitable ways in accordance with further embodiments.

The hinge assembly 190 can allow the first bar 110 and second bar 160 to be rotated relative to each other such that the member cavity 195 can be opened and closed. For example, the incontinence clamp 100 can assume an open configuration where the stop tip 176 is separated from the stop notch 136 and/or the ratchet tooth 178 is disengaged from the ratchet slots 134 of the ratchet end 130 of the first bar 110. In such a configuration, the first and second front arms 116, 166 can be separated such that objects (e.g., a male member) can be inserted into the member cavity 195, such as between the ends of the first and second front arms 116, 166 and/or sides of the incontinence clamp 100.

In various embodiments, the first bar 110 and second bar 160 can be rotated together and releasably coupled together. For example, the first bar 110 and second bar 160 can be rotated towards each other and pressed together (e.g., with fingers of a user) such that the ratchet tooth 178 clicks into the first rachet slot 134A (See FIG. 2c) based at least on flexion of the ratchet end 130 of the first bar 110 that biases the ratchet end 130 toward the ratchet tooth 178 such that the ratchet tooth 178 is held within the first rachet slot 134A such that the first bar 110 and second bar 160 are releasably coupled together at the ends of the first and second front arms 116, 166 of the first and second bars 110, 160.

To further close the first and second bars 110, 160 together and reduce the volume of the member cavity 195, the first and second bars 110, 160 can be pressed together (e.g., with fingers of a user) such that the ratchet tooth 178 clicks into the second rachet slot 134B, then into the third rachet slot 134C and then into the fourth rachet slot 134D based at least on flexion of the ratchet end 130 of the first bar 110 that biases the ratchet end 130 toward the ratchet tooth 178 such that the ratchet tooth 178 is held within the rachet slots 134 such that the first bar 110 and second bar 160 are releasably coupled together at the ends of the first and second front arms 116, 166 of the first and second bars 110, 160.

In various embodiments, the stop tip 176 at the end of the second front arm 166 can be configured to engage with the stop notch 136 of the ratchet end 130 of the first bar 110 to generate a stop between the first bar 110 and the second bar 160 that prevents the first and second bars 110, 160 from moving closer together and/or past the fourth rachet slot 134D.

In various embodiments, the planar and parallel second bar external and internal faces 170, 172 defined by second bar central body 162 can be disposed in parallel to the first bar internal face 122 when the ratchet tooth 178 is in the last ratchet slot 134 (e.g., fourth ratchet slot 134D) and/or when the stop tip 176 at the end of the second front arm 166 engages the stop notch 136.

To disengage the first and second front arms 116, 166 of the first and second bars 110, 160, in various embodiments a user can lift the ratchet end 130 of the first bar 110 (e.g., via the first arm tip 132), which can disengage the ratchet tooth 178 from the fourth rachet slot 134D or other slot that the ratchet tooth 178 may be disposed in. This can allow the first and second front arms 116, 166 of the first and second bars 110, 160 to be separated to expand the member cavity 195.

In various embodiments, a portion of the ratchet end 130 (e.g., the first arm tip 132 and/or one or more ratchet slots 134) can extend below past a plane of one or both of internal and/or external faces 170, 172, which may be desirable in some examples to provide access to the ratchet end 130 such that a user can actuate the ratchet end 130 (e.g., with their finger(s)) to disengage the first and second front arms 116, 166 of the first and second bars 110, 160. Accordingly, in various embodiments the first bar 110 can comprise any suitable material and/or structure that provides for such living flexibility of the ratchet end 130 and/or first front arm 116. In various embodiments, the first and second bars 110, 160 can be made of any suitable material such as a plastic, metal, wood, or the like.

While various embodiments can provide for a releasable coupling between the first and second front arms 116, 166 of the first and second bars 110, 160, various suitable elements can be used to provide such a releasable coupling in further embodiments, including friction fit elements, hook and loop tape, a magnet, a snap, a pin, an adhesive, a buckle, a clamp, a clip, an elastic band, or the like.

In various embodiments, the incontinence clamp 100 can be various suitable sizes including in one preferred embodiment, where the incontinence clamp 100 has a maximum width of 16mm, a maximum length of 72.1mm, and a maximum height between the external faces 120, 170 of 32.7mm. In some embodiments the incontinence clamp 100 can have a maximum width of 12mm, 13mm, 14mm, 15mm16mm, 17mm, 18mm, 19mm, 20mm, or the like, or a range between such example values. In some embodiments the incontinence clamp 100 can have a maximum length of 65mm, 66mm, 67mm, 68mm, 69mm, 70mm, 71mm, 72mm, 73mm, 74mm, 75mm, 76mm, 77mm, 78mm, 79mm, and the like, or a range between such example values. In some embodiments the incontinence clamp 100 can have a maximum height between the external faces 120, 170 of 22mm, 23mm, 24mm, 25mm, 26mm, 27mm, 28mm, 29mm, 30mm, 31mm, 32mm, 33mm, 34mm, 35mm, 36mm, 37mm, 38mm, 39mm, 40mm, or the like or a range between such example values.

Turning to FIGS. 3, 4, and 5, examples of an adjustment assembly 140 are illustrated, including an exploded view of an adjustment assembly 140 in FIG. 3 which illustrates that an adjustment assembly 140 can comprise an adjustment cylinder 342 with a key flange 344 (see FIGS. 4a and 4b), an extension shaft 346, an adjustment plate 348, and a first pad retainer 350 is part of the first pad assembly 150.

In various embodiments, portions of the adjustment assembly 140 can be disposed with an adjustment assembly cavity 128 defined by the first bar central body 112 of the first bar 110. For example, as shown in the embodiments of FIGS. 3, 4, and 5, the adjustment cylinder 342 can be disposed generally flush or below flush with the first bar external face 120 and extending into the adjustment assembly cavity 128. The extension shaft 346 can be disposed within the adjustment assembly cavity 128 and can rotatably couple with the extension shaft 346 within the adjustment assembly cavity 128.

The adjustment plate 348 can couple over the opening of the adjustment assembly cavity 128 at the first bar internal face 122 and define a portion of the first bar internal face 122. The extension shaft 346 can extend through the adjustment plate 348 and past the face of the first bar internal face 122 and the extension shaft 346 can be configured to extend and retract based on turning of the adjustment cylinder 342. For example, FIGS. 4a and 4b illustrate an embodiment of the adjustment cylinder 342 in a first and second configuration respectively (i.e., turned 90 degrees), which causes the extension shaft 346 to extend and retract past the face of the first bar internal face 122.

The first pad retainer 350 can be disposed and coupled within a slot of the first pad 150A and the first pad retainer 350 can be retained by elements of the adjustment plate 348 such that the first pad retainer 350 holds the first pad 150A at the first bar internal face 122. In various embodiments, the extension shaft 346 can be configured to extend and retract past the face of the first bar internal face 122 and within the first pad retainer 350 to press on the first pad 150A, which can cause the flexible first pad 150A to distend into the member cavity 195, become more rigid, or the like. As discussed in more detail herein, this can apply a suitable and desired amount of pressure to a male member for the treatment of incontinence. However, in some embodiments, the extension shaft 346, or other suitable element, can be configured to move the first pad 150A (e.g., such that the first pad 150A extends away from the first bar internal face 122).

In various embodiments, the extension shaft 346 can be configured to extend and retract a between a minimum and maximum distance of 0.5mm, 0.75mm, 1.0mm, 1.25mm, 1.5mm, 1.75mm, 2.0mm, or the like, or a range between such example values. In some embodiments, the extension shaft 346 can be configured to extend and retract between a minimum and maximum distance of 1.0mm, 2.0mm, 3mm, 4mm, 5mm, 6mm, 7mm, or the like, or a range between such example values.

Turning to FIGS. 4a and 4b, an example of rotating the adjustment cylinder 342 to configure extension and retraction of the extension shaft 346 between a minimum and maximum extension distance. Here, the adjustment cylinder 342 is shown being rotated 90 degrees to cause extension and retraction of the extension shaft 346 between a minimum and maximum extension distance. For example, in various embodiments a key 300 (see FIG. 3) can be used to rotate the adjustment cylinder 342, with the key comprising a head 302, a base 304 and a key slot 306 defined by the base 304. To rotate the adjustment cylinder 342 back and forth, a user can engage the key flange 344 in the key slot 306 and turn the key 300 to rotate the adjustment cylinder 342 a desired amount (e.g., between or equal to 0 degrees and 90 degrees as shown in the example of FIGS. 4a and 4b).

In various embodiments, it can be desirable for the key 300 to be separate from the adjustment cylinder 342 so that the male incontinence clamp 100 is not bulky when being worn by a user. In other words, in various embodiments, it can be desirable to have a key 300 with a large head 302 that can be easily manipulated by a user to configure the adjustment cylinder 342, but without the key 300 being integral to the male incontinence clamp 100 and making the male incontinence clamp 100 bulky while being worn by the user.

However, in further embodiments, the fit of the male incontinence clamp 100 can be configured in any suitable way, and the examples herein should not be construed as being limiting. For example, in some embodiments, an adjustment cylinder 342 can extend above the first bar external face 120, a key 300 or similar elements can be integral to the adjustment cylinder 342, or the like.

Also, while fit of the male incontinence clamp 100 can be configured via a rotatable element such as an adjustment cylinder 342, comprising a screw, threads, tabs, or the like, further embodiments can include any suitable element(s) to adjust the fit of the male incontinence clamp 100 such as a slider, ratchet, knock mechanism, or the like. Also, while various embodiments include an element such as an extension shaft 346, further embodiments can be configured to adjust the fit of the first and/or second pad assembly 150, 180, or portions thereof, in various suitable ways, so the examples herein should not be construed as being limiting.

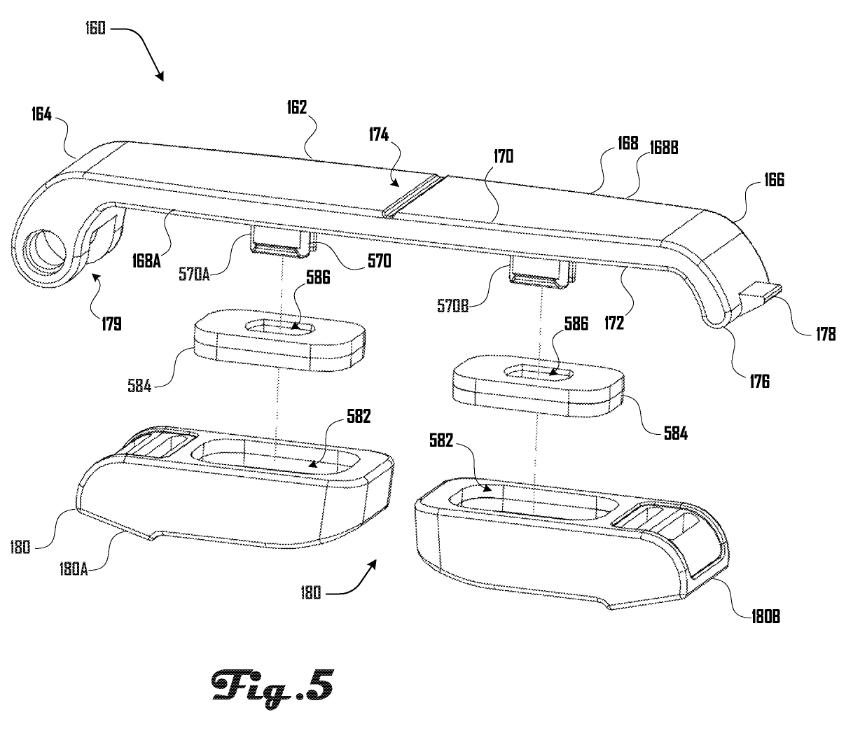

In various embodiments, the first and second pads 180A, 180B of the second pad assembly 180 can be coupled to the second bar 160 in various suitable ways. For example, as shown in FIGS. 5 and 6, the first and second pads 180A, 180B can comprise respective coupling holes 582, in which a respective pair of retaining plates 584 can be disposed. For example, the first and second pads 180A, 180B can be flexible and can hold the pair of retaining plates 584 (which can be rigid in various examples), via friction fit in the coupling holes 582. In various embodiments, the first and second pads 180A, 180B can comprise a flexible material such as silicone, rubber, or the like.

The second bar 160 can comprise a first and second retaining tab 570A, 570B that extend from the internal face 172 of the second bar 160, which can couple within respective retaining slots 586 defined by the respective retaining plates 584. Accordingly, in various embodiments, the first and second pads 180A, 180B can be removably coupled to the second bar 160 via the retaining plates 584 coupling to the retaining tabs 570.

In various embodiments, the second pad assembly 180 (e.g., comprising first and second pads 180A, 180B, or the like), can be integrally or removably coupled to the second bar 160 in various suitable ways, so the examples herein should not be construed as limiting. For example, in some embodiments, the second pad assembly 180 (e.g., comprising first and second pads 180A, 180B, or the like), can be coupled to the second bar 160 via one or more of welding, an adhesive, hook and loop tape, a flange, a slot, friction fit, or the like.

As discussed herein, the male incontinence clamp 100 can be configured to address incontinence (e.g., unintentional leakage of urine from the bladder through the penis) of a user by applying the male incontinence clamp 100 along the shaft of the penis such as between the base and head of the penis. The male incontinence clamp 100 can be configured in various embodiments to address incontinence in the user while also providing comfort and safety for the user.

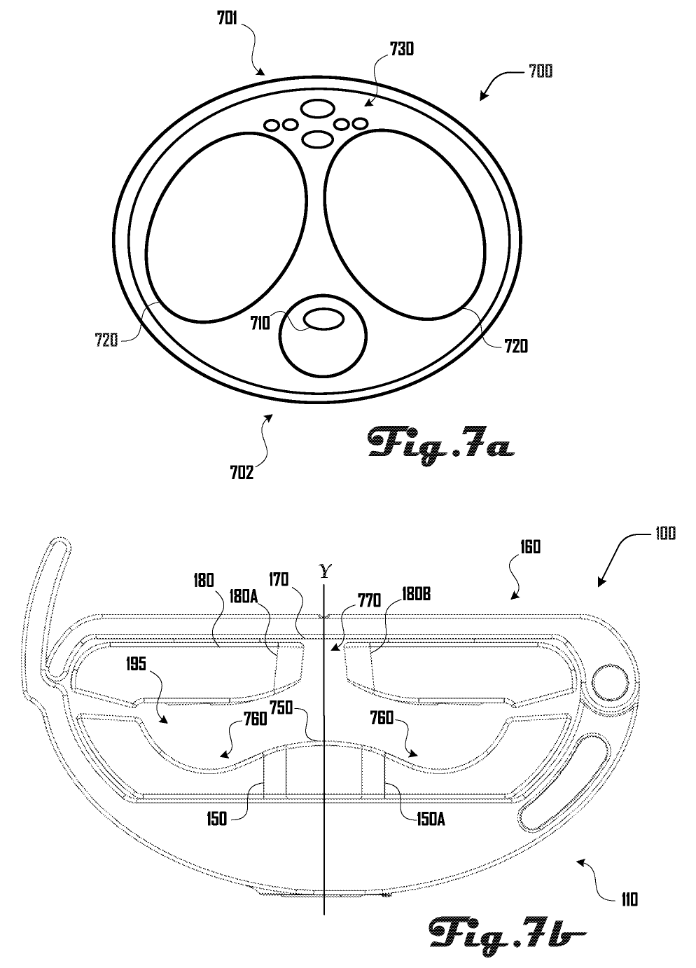

For example, FIG. 7a illustrates an example anatomical cross section of a male member 700 (i.e., penis), and FIG. 7b illustrates for reference a side view of a male incontinence clamp 100. Note that these figures may not be drawn in the same scale and FIG. 7a may be drawn larger than FIG. 7b so that the details of FIG. 7a can be clear.

As shown in FIG. 7, the male member 700 comprises a dorsal end 701 (i.e., top end) and a ventral end 702 (i.e., bottom end). Centrally located at the ventral end 702 is the urethra 710 and between the dorsal and ventral ends 701, 702 are a pair of corpus cavernosum 720. Centrally located at the dorsal end is a dorsal neurovascular bundle 730 that comprises a set of veins, arteries and nerves that run along the dorsal end 701 of the male member 700.

In various embodiments, the first pad assembly 150 and the second pad assembly 180 (e.g., comprising first and second pads 180A, 180B, or the like) and the member cavity 195 defined thereby can be configured for the anatomy of the male member 700 to address incontinence in the user while also providing comfort and safety for the user. For example, as shown in the example of FIG. 7b, the first pad assembly 150 can comprise a convex central ridge 750 and a pair of concave peripheral valleys 760 on opposing sides of the central ridge 750. In various embodiments, the convex central ridge 750 can have continuous sloping connection to the pair of concave peripheral valleys 760 on opposing sides of the central ridge 750, which can be desirable in some examples for user comfort for lack of edges, or the like. In various embodiments, the first pad assembly 150 can have central symmetry, such as about a central axis Y of the male incontinence clamp 100 in various configurations.

The second pad assembly 180 comprising first and second pads 180A, 180B can be disposed symmetrically on opposing sides of the central axis Y of the male incontinence clamp 100 and define a member gap 770, which can be a portion of the member cavity 195 defined at least in part by the first and second pads 180A, 180B and first pad assembly 150. The ends of the first and second pads 180A, 180B facing the member gap 770 can slope to make the first and second pads 180A, 180B thicker farther from such central ends.

In various embodiments, the second pad assembly 180 comprising first and second pads 180A, 180B and/or first pad assembly 150 can have symmetry about the central axis Y such that views as in FIGS. 3 and 5 would be the same at the corresponding three other perspective angles at 90 degrees, 180 degrees and 270 degrees. Accordingly, the full physical external shape of the second pad assembly 180 comprising first and second pads 180A, 180B and/or first pad assembly 150 in various embodiments is fully enabled based on the present disclosure including based on the specification and/or drawings herein.

As discussed herein, in various embodiments, a male member 700 can be disposed within the member cavity 195 such that the male member 700 is centrally aligned along the axis Y of the male incontinence clamp 100 with the same orientations as shown in FIGS. 7a and 7b. The member cavity 195 can be sized such that a male member 700 clamped within the male incontinence clamp 100 experiences sufficient compression on the urethra 710 of the male member 700 to address incontinence by preventing urine from leaking from the bladder through the urethra 710. For example, the convex central ridge 750 can be aligned with the urethra 710 to apply pressure to the urethra 710 with the pair of peripheral valleys 760 on opposing sides of the central ridge 750 configured to be located and/or shaped corresponding to the corpus cavernosum 720 such that the corpus cavernosum 720 generally reside within the peripheral valleys 760. Such a configuration can be desirable because it can allow the corpus cavernosum 720 to be safely and comfortably held in the peripheral valleys 760, while the convex central ridge 750 applies sufficient pressure to the urethra 710, which in some examples can be pressed against or proximate to the corpus cavernosum 720.

Additionally, the male member 100 can be positioned such that one or more elements of the dorsal neurovascular bundle 730 are disposed within the member gap 770 and/or on sloping central sides of the first and second pads 180A, 180B. Peripheral side portions of the dorsal end 701 of the male member 700 can be supported by the faces of the first and second pads 180A, 180B. Such a configuration can be desirable to provide sufficient opposing support to the dorsal end 701 of the male member 700 while providing minimal pressure or compression on the elements of the dorsal neurovascular bundle 730. For example, providing minimal pressure on the veins, arteries and/or nerves that run along the dorsal end 701 of the male member 700 can be desirable because it can provide for enhanced comfort and safety for the user by minimizing or preventing the blockage of blood flow through the veins and/or arteries of the dorsal neurovascular bundle 730 and/or by minimizing or preventing pressure on nerves of the dorsal neurovascular bundle 730 that may cause discomfort, nerve damage, or the like.

Additionally, pressure on the urethra 710 of the male member 700 can be fine tuned to provide a desirable balance between comfort to the user and sufficient pressure on the urethra 710 to prevent, reduce or otherwise address incontinence. For example, as discussed herein, the extension shaft 346 can be configured to extend and retract past the face of the first bar internal face 122 and within the first pad retainer 350 to press on the first pad assembly 150, which can cause the flexible first pad 150A to distend into the member cavity 195, become more rigid, or the like to generate additional pressure on the urethra 710 of the male member 700 at the ventral end 702 of the male member 700.

Accordingly, male incontinence clamp 100 in various embodiments can be configured and sized in various embodiments with a member cavity 195 defined at least in part by first and second pads 180A, 180B and first pad assembly 150 and/or adjustment assembly 140 to address incontinence in the user while also providing comfort and safety for the user based on the anatomy of the male member 100 of the user 100.

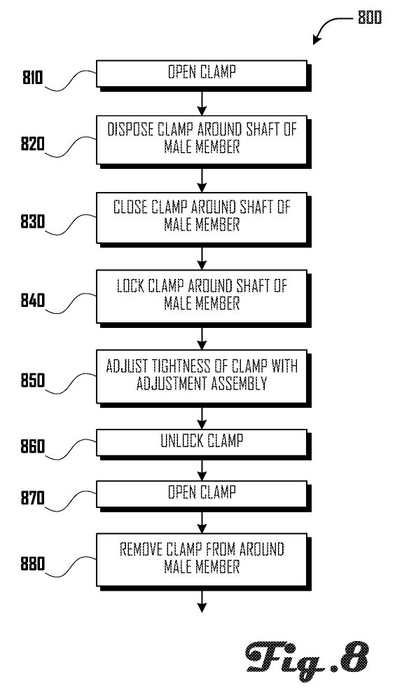

Turning to FIG. 8, a method 800 of using a male incontinence clamp 100 is illustrated, which begins at 810, where the male incontinence clamp 100 is opened. For example, in various embodiments, opening the male incontinence clamp 100 can include the user grasping and pulling the first and second bars 110, 160 apart via the rotatable coupling of the hinge assembly 190 to separate the first and second front arms 116, 166 to create an opening therebetween. In some embodiments, opening the male incontinence clamp 100 can comprise unlocking the male incontinence clamp 100 as discussed herein.

Returning to the method 800 the male incontinence clamp 100 is disposed around the shaft of a male member 700. For example, the tip of the male member 700 can be inserted into the member cavity 195 through one of the sides or through the opening between the first and second front arms 116, 166 discussed above. Additionally, in various embodiments, the male incontinence clamp 100 can be oriented on the male member 700 with a central axis of the male member 700 disposed coincident with a central axis Y of the male incontinence clamp 100 (see e.g., FIGS. 7a and 7b and associated discussion). In some examples, the dorsal gap 174 can be used to orient the male incontinence clamp 100 centrally on the dorsal end 701 of the male member 700, given that in various embodiments, the dorsal gap 174 is coincident with the central axis Y of the male incontinence clamp 100. As discussed herein, such an orientation can be desirable for addressing incontinence of the user while maximizing comfort and safety of the user while wearing the male incontinence clamp 100.

Returning to the method 800, the male incontinence clamp 100 can be closed around the shaft of the male member 700, at 830, and locked around the shaft of the male member 700 at 840. For example, the first and second bars 110, 160 can be pushed together via the rotatable coupling of the hinge assembly 190 to close the opening therebetween, and closing the first and second bars 110, 160 can cause the ratchet tooth 178 to click into the first rachet slot 134A (See e.g., FIG. 2c) based at least on flexion of the ratchet end 130 of the first bar 110 that biases the ratchet end 130 toward the ratchet tooth 178 such that the ratchet tooth 178 is held within the first rachet slot 134A such that the first bar 110 and second bar 160 are releasably coupled together at the ends of the first and second front arms 116, 166 of the first and second bars 110, 160. In various embodiments, the ratchet end 130 is biased to click into the ratchet slots 134 without elements such as springs, or the like and solely based on flexibly of the first and/or second arms 110, 160.

To further close the first and second bars 110, 160 together and reduce the volume of the member cavity 195, in various embodiments the first and second bars 110, 160 can be pressed together (e.g., with fingers of a user) such that the ratchet tooth 178 clicks into the second rachet slot 134B, then into the third rachet slot 134C and then into the fourth rachet slot 134D based at least on flexion of the ratchet end 130 of the first bar 110 that biases the ratchet end 130 toward the ratchet tooth 178 such that the ratchet tooth 178 is held within the rachet slots 134 such that the first bar 110 and second bar 160 are releasably coupled or locked together at the ends of the first and second front arms 116, 166 of the first and second bars 110, 160. In some embodiments, the user may choose to not fully close the male incontinence clamp 100 (e.g., to a final ratchet slot 134, such as the fourth rachet slot 134D in some example), and may instead choose another rachet slot 134 to stop at based on desired or necessary fit to address incontinence while having sufficient comfort and safety. In various embodiments, orientation of the male incontinence clamp 100 on the shaft of the male member 700 as discussed herein can be performed as the male incontinence clamp 100 is being closed and/or locked about the male member 700.

Returning to the method 800, at 850, the tightness of the male incontinence clamp 100 can be adjusted via an adjustment assembly 140. For example, in some embodiments, the user can use a key 300 (see FIG. 3) to rotate an adjustment cylinder 342 back and/or forth, to cause an extension shaft 346 to extend and/or retract to press on the first pad 150A, which can cause the flexible first pad 150A to distend into the member cavity 195 against the male member 700, become more rigid, or the like, which can adjust the pressure against the male member 700. To apply a suitable, desired or necessary amount of pressure to the male member for the treatment of incontinence and/or to provide a suitable, desired or necessary amount of comfort to the user wearing the male incontinence clamp 100. In various embodiments, the first pad 150A and/or portions of the first pad assembly 150 can comprise a flexible material such as silicone, rubber, or the like.

In various embodiments, it can be desirable for the key 300 to be separate from the adjustment cylinder 342 so that the male incontinence clamp 100 is not bulky when being worn by a user, which may include while wearing clothing in some examples. For example, in various embodiments, it can be desirable to have a key 300 with a large head 302 that can be easily manipulated by the fingers of a user to configure the adjustment cylinder 342, but without the key 300 being integral to the male incontinence clamp 100 and making the male incontinence clamp 100 bulky while being worn by the user such as in pants, shorts, or the like.

Returning to the method 800, at 860, the male incontinence clamp 100 can be unlocked; the male incontinence clamp 100 can be opened at 870; and the male incontinence clamp 100 can be removed from around the male member 700 at 880. For example, a user wearing the male incontinence clamp 100 can choose to remove or adjust the position of the male incontinence clamp 100 along the male member 700, which may be desirable for the user to relieve pressure on the male member 700, which can be to prevent over-exposure to pressure on nerves, veins, arteries, and the like, in the male member 700. In some examples, it can be desirable to periodically change the position of the male incontinence clamp 100 on the male member 700 to prevent damage or discomfort to the male member 700.

Accordingly, to remove and/or reposition the male member, the user can unlock the male incontinence clamp 100 to assume an open configuration where the stop tip 176 is separated from the stop notch 136 and/or the ratchet tooth 178 is disengaged from the ratchet slots 134 of the ratchet end 130 of the first bar 110. This can be done in some embodiments by pulling on the first arm tip 132 of the ratchet end 130 of the first bar 110 to release the ratchet tooth 178 from one or more ratchet slots 134 (e.g., based on flexible rigidity of the first bar 110). In such a configuration, the first and second front arms 116, 166 can be separated such that the male member 700 can be removed from or adjusted within the member cavity 195, such as between the ends of the first and second front arms 116, 166 and/or sides of the incontinence clamp 100.

The described embodiments are susceptible to various modifications and alternative forms, and specific examples thereof have been shown by way of example in the drawings and are herein described in detail. It should be understood, however, that the described embodiments are not to be limited to the particular forms or methods disclosed, but to the contrary, the present disclosure is to cover all modifications, equivalents, and alternatives. Additionally, elements of a given embodiment should not be construed to be applicable to only that example embodiment and therefore elements of one example embodiment can be applicable to other embodiments. Additionally, elements that are specifically shown in example embodiments should be construed to cover embodiments that comprise, consist essentially of, or consist of such elements, or such elements can be explicitly absent from further embodiments. Accordingly, the recitation of an element being present in one example should be construed to support some embodiments where such an element is explicitly absent.

What is claimed is:

1. A method of using a male incontinence clamp to address incontinence in a user, the method comprising:
increasing the volume of a member cavity of the male incontinence clamp, the male incontinence clamp comprising:
a first bar that includes a first bar internal face and a first bar external face,
a second bar comprising a second bar internal face and a second bar external face,
a rotatable adjustment assembly coupled to and extending through the second bar, the rotatable adjustment assembly comprising a screw with threads and configured to extend and retract away from and toward the internal face of the second bar and into the member cavity, by rotating at least a portion of the rotatable adjustment assembly,
the member cavity defined at least in part by the first bar and the rotatable adjustment assembly, wherein a portion of the rotatable adjustment assembly and the first bar are configured to engage a male member disposed within the member cavity, and
one or more sliders configured to adjust the fit of the male incontinence clamp including allowing the volume of the member cavity to be increased and decreased,
wherein increasing the volume of the member cavity of the male incontinence clamp includes configuring the one or more sliders to increase the volume of the member cavity;
inserting the male member into the member cavity defined at least in part by the first bar and the rotatable adjustment assembly;
adjusting the fit of the male incontinence clamp via the one or more sliders while the male member is within the member cavity;
adjusting the fit of the male incontinence clamp via the rotatable adjustment assembly, by rotating the rotatable adjustment assembly while the male member is within the member cavity, wherein adjusting the fit of the male incontinence clamp via the one or more sliders and via the rotatable adjustment assembly causes:
the male incontinence clamp to be coupled to the male member, and
the male incontinence clamp to apply sufficient pressure to a urethra of the male member to address incontinence associated with the male member of the user; and
de-coupling the incontinence clamp from the male member and removing the male member from the member cavity by at least one of:
adjusting the fit of the male incontinence clamp via the one or more sliders while the male member is within the member cavity, and
adjusting the fit of the male incontinence clamp via the rotatable adjustment assembly, by rotating the rotatable adjustment assembly while the male member is within the member cavity.

2. The method of using a male incontinence clamp of claim 1, wherein the first bar has an arched shape.

3. The method of using a male incontinence clamp of claim 1, wherein the second bar is planar.

4. The method of using a male incontinence clamp of claim 1, wherein the first and second bars are made of plastic.

5. A method of using a male incontinence clamp to address incontinence in a user, the method comprising:
applying the male incontinence clamp to a male member of the user, the male incontinence clamp comprising:
a first bar that includes a first bar internal face,
a second bar comprising a second bar internal face and a second bar external face,
a rotatable adjustment assembly coupled to and extending through the second bar, the rotatable adjustment assembly comprising a screw with threads and configured to extend and retract away from and toward the internal face of the second bar by rotating at least a portion of the rotatable adjustment assembly,
a member cavity defined at least in part by the first bar and the rotatable adjustment assembly, wherein a portion of the rotatable adjustment assembly and the first bar are configured to engage the male member that is disposed within the member cavity, and
one or more sliders configured to adjust the fit of the male incontinence clamp including allowing the volume of the member cavity to be increased and decreased;
adjusting the fit of the male incontinence clamp via the one or more sliders while the male member is within the member cavity; and
adjusting the fit of the male incontinence clamp via the rotatable adjustment assembly, by rotating the rotatable adjustment assembly while the male member is within the member cavity, wherein adjusting the fit of the male incontinence clamp via the one or more sliders and/or via the rotatable adjustment assembly causes:
the male incontinence clamp to be coupled to the male member, and
the male incontinence clamp to apply sufficient pressure to a urethra of the male member to address incontinence associated with the male member of the user.

6. The method of using a male incontinence clamp of claim 5, further comprising increasing the volume of the member cavity of the male incontinence clamp by configuring the one or more sliders to increase the volume of the member cavity.

7. The method of using a male incontinence clamp of claim 6, wherein increasing the volume of the member cavity of the male incontinence clamp by configuring the one or more sliders to increase the volume of the member cavity allows the user insert the male member into the member cavity defined at least in part by the first bar and the rotatable adjustment assembly.

8. The method of using a male incontinence clamp of claim 5, further comprising:
de-coupling the male incontinence clamp from the male member and removing the male member from the member cavity by at least one of:
adjusting the fit of the male incontinence clamp via the one or more sliders while the male member is within the member cavity, and
adjusting the fit of the male incontinence clamp via the rotatable adjustment assembly, by rotating the rotatable adjustment assembly while the male member is within the member cavity.

9. The method of using a male incontinence clamp of claim 5, wherein the first bar has an arched shape and wherein the second bar is planar.

10. A method of using a male incontinence clamp to address incontinence in a user, the method comprising:
applying the male incontinence clamp to a male member of the user, the male incontinence clamp comprising:
a first bar,
a second bar,
a rotatable adjustment assembly coupled to the second bar,
a member cavity, and
one or more sliders configured to adjust the fit of the male incontinence clamp including allowing the volume of the member cavity to be increased and decreased.

11. The method of using a male incontinence clamp of claim 10, wherein the first bar includes at least a first bar internal face.

12. The method of using a male incontinence clamp of claim 10, wherein the second bar includes a second bar internal face and a second bar external face.

13. The method of using a male incontinence clamp of claim 10, wherein the rotatable adjustment assembly is coupled to and extends through the second bar.

14. The method of using a male incontinence clamp of claim 10, wherein the rotatable adjustment assembly comprises a screw with threads.

15. The method of using a male incontinence clamp of claim 10, wherein the rotatable adjustment assembly is configured to extend and retract away from and toward an internal face of the second bar by rotating at least a portion of the rotatable adjustment assembly.

16. The method of using a male incontinence clamp of claim 10, wherein the member cavity is defined at least in part by the first bar and by the rotatable adjustment assembly, wherein a portion of the rotatable adjustment assembly and the first bar are configured to engage the male member.

17. The method of using a male incontinence clamp of claim 10, further comprising adjusting the fit of the male incontinence clamp via the one or more sliders while the male member is within the member cavity.

18. The method of using a male incontinence clamp of claim 10, further comprising adjusting the fit of the male incontinence clamp via the rotatable adjustment assembly, by rotating the rotatable adjustment assembly while the male member is within the member cavity.

19. The method of using a male incontinence clamp of claim 10, wherein adjusting the fit of the male incontinence clamp via the one or more sliders and/or via the rotatable adjustment assembly causes:
the male incontinence clamp to be coupled to the male member, and
the male incontinence clamp to apply sufficient pressure to a urethra of the male member to address incontinence associated with the male member of the user.

20. The method of using a male incontinence clamp of claim 10, wherein the first bar has an arched shape and wherein the second bar is planar.
